(19) United States
(12) Patent Application Publication
(10) Pub. No.: US 2010/0015069 A1
(43) Pub. Date: Jan. 21, 2010

(54) Bi-component oral treatment composition

(76) Inventor: Giuseppe Bosco, Palermo (IT)

Correspondence Address: Themis Law, 7660 Fay Ave Ste H-535, La Jolla, CA, 92037, US

(21) Appl. No.: 12/443,725

(22) PCT Filed: Oct. 9, 2007
(86) PCT No.: PCT/IB07/54108

§ 371 Date: Mar. 31, 2009

(30) Foreign Application Priority Data
Oct. 10, 2006 (IT) PA2006U000020
Jul. 31, 2007 (IT) SMA200700031

(51) Int. Cl. A61K 8/97; A61K 8/18; A61Q 11/00

(52) U.S. Cl. 424/58; 424/49

(57) ABSTRACT

An oral hygiene composition comprises a first acidic liquid component and a second basic liquid component, for separate use by a user. The first acidic component comprises more than 60% by weight of a solution of an acidic liquid food, based on the total weight of the component, at least one first essential oil and an orally acceptable liquid carrier. The second basic component comprises less than 15% by weight of a solution of at least one basic salt, based on the total weight of the second component, at least one second essential oil and an orally acceptable liquid carrier.

### FIELD OF THE INVENTION

The present invention finds application in the field of compositions for treatment of the oral cavity, and particularly relates to a two-component composition as defined in the preamble of claim 1.

The invention further relates to the use of such composition for hygienic treatment of the oral cavity, and a kit comprising such composition.

### BACKGROUND OF THE INVENTION

Good oral hygiene practices are known to require a number of measures and behaviors to ensure hygiene of the oral cavity. As is known, mouthwashes have a refreshing, deodorizing and cosmetic action.

Currently available mouthwashes include a single liquid component, that is used by a user for mouth rinsing one or more times. This liquid component generally consists of a solution of multiple treating and flavoring agents.

In practice, these prior art mouthwashes have been found to be ineffective in maintaining oral hygiene, and even more in ensuring a refreshing and deodorizing action.

### SUMMARY OF THE INVENTION

The object of the present invention is to overcome the above drawbacks, by providing a two-component composition that is highly efficient and relatively cost-effective.

A particular object is to provide a two-component composition that ensures an effective refreshing, deodorizing and cosmetic action.

Another object of the invention is to provide a composition that is effective in maintaining oral hygiene.

These and other objects, as better explained hereafter, are fulfilled by a composition as defined in claim 1, comprising a first acidic liquid component and a second basic liquid component designed for separate use by a user, characterized in that the first acidic component comprises more than 60% by weight of a solution of an acidic liquid food, based on the total weight of the first component, at least one first essential oil and an orally acceptable liquid carrier, and in that the second basic component comprises less than 15% by weight of a solution of at least one basic salt, based on the total weight of the second component, at least one second essential oil and an orally acceptable liquid carrier.

The composition of the invention has been found to be particularly effective in maintaining oral hygiene, and even more in ensuring a refreshing and deodorizing cosmetic action.

As used herein, the term “acidic liquid component” will be intended as liquid phase at ambient temperature and pressure having a pH of 2.5 to 7.

Also, as used herein, the term “basic liquid component” will be intended as liquid phase at ambient temperature and pressure having a pH of 7 to 10.

Advantageously, the acidic liquid food may include at least one acid selected from the group composed of citric acid, malic acid, tartaric acid, oxalic acid, succinic acid, fumaric acid, acetic acid, amyl acid, or a combination thereof. Its content in the first component is 65% to 75% by weight, based on its total weight, and is preferably about 70%.

Suitably, the basic salt content in the second component may be 5% to 10% by weight, based on the total weight of the second component, and is preferably about 7.5%.

Also, preferably the second essential oil content may be 0.005% to 1% by weight, based on the total weight of the second component, and is preferably about 0.15%.

Suitably, the first essential oil and the second essential oil respectively may be selected from the group composed of essential oils extracted from: anise, orange, basil, bergamot, birch, cajeput, chamomile, camphor, cinnamon, cedarwood, cypress, citronella, eucalyptus, fennel seeds, clove leaves, geranium, juniper berry, incense, hyssop, lavender, hybrid lavender, lemongrass, lemon, marjoram, malaleuca alternifolia, tangerine, melissa, mint, myrrh, oregano, patchouli, scotch pine, mugo pine, grapefruit, rose, rosemary, sage, sandalwood, thyme, ylang-ylang, ginger, or a mixture thereof. Also, its content may be 0.01% to 1% by weight, based on the total weight of the first component, and preferably about 0.1%.

In another aspect, the invention relates to the use of the above composition for cosmetic treatment of the oral cavity, as defined in claim 11.

In yet another aspect, the invention relates to a kit for oral hygiene as defined in claim 14, characterized in that it comprises a first container for the first acidic liquid component as described above, and a second container for the second basic liquid component as described above.

Advantageous embodiments of the invention are as defined in the dependent claims.

Further features and advantages of the invention will be more apparent with reference to the following preferred, non exclusive embodiments of the composition of the invention.

### Example 1

The first acidic liquid component is a solution of the following compounds:
69.9% lemon juice;
0.1% orange essential oil;
30% distilled water.

The solution is prepared at ambient temperature and pressure, with methods known to those of ordinary skill in the art.

The second basic liquid component is a solution of the following compounds:
7.7% sodium bicarbonate;
0.019% thyme essential oil;
0.055% eucalyptus essential oil;
0.055% mint essential oil;
92.171% distilled water.

The solution is prepared at ambient temperature and pressure, with methods known to those of ordinary skill in the art.

The two components of the composition so prepared will be placed in respective containers, a first container for the first component and a second container for the second component, which are designed for separate use by a user.

To ensure an effective cosmetic oral cavity deodorizing and refreshing action, the following method of use may be used:

The user will first draw a predetermined amount of first component from a first container and proceed to a first oral cavity rinsing step for a predetermined time, after which the first component will be ejected.

Then, the user will draw a predetermined amount of second component from a second container and proceed to a second rinsing step for a predetermined time, after which the second component will be ejected.

For maximized effectiveness, the second rinse must necessarily follow the first rinse.

An amount of 5 ml to 20 ml, preferably of about 15 ml, may be used for each of the first and second rinses. The first and second rinses may each last 20 seconds to 40 seconds, and preferably about 30 seconds.

### Example 2

The first acidic liquid component consists of a solution of the following compounds:
67% apple vinegar;
0.05% bergamot essential oil;
0.07% tangerine essential oil;
32.88% distilled water.

The solution is prepared at ambient temperature and pressure, with methods known to those of ordinary skill in the art.

The second basic liquid component is a solution of the following compounds:
7.5% sodium bicarbonate;
0.025% mugo pine essential oil;
0.07% juniper berry essential oil;
0.08% fennel seed essential oil;
92.325% distilled water.

The solution is prepared at ambient temperature and pressure, with methods known to those of ordinary skill in the art.

Packaging and use of the composition may be as described in Example 1.

### Example 3

The first acidic liquid component consists of a solution of the following compounds:
37% currant juice;
32% lemon juice;
0.08% cinnamon essential oil;
30.92% distilled water.

The solution is prepared at ambient temperature and pressure, with methods known to those of ordinary skill in the art.

The second basic liquid component is a solution of the following compounds:
7.2% sodium bicarbonate;
0.06% oregano essential oil;
0.03% sage essential oil;
92.71% distilled water.

The solution is prepared at ambient temperature and pressure, with methods known to those of ordinary skill in the art.

Packaging and use of the composition may be as described in Example 1.

The above disclosure clearly shows that the two-component composition of the invention fulfils the intended objects, and particularly meets the requirement of ensuring an effective cleansing, refreshing and deodorizing action.

The composition of the invention has also proved to be particularly effective in maintaining a correct oral hygiene.

The composition of the invention has been further found to be effective in other physiological situations of in which the physiological pH is altered.

What is claimed is:

1. A composition for oral hygiene comprising
a first acidic liquid component; and
a second basic liquid component,
wherein said first acidic liquid component and said second basic liquid component are designed for separate use,
wherein said first acidic liquid component consists of a solution including an acidic liquid food, at least one first essential oil and an orally acceptable liquid carrier, said acidic liquid food being more than 60% by weight, based on the total weight of said first acidic liquid component, and
wherein said second basic liquid component consists of a solution including at least one basic salt, at least one second essential oil and an orally acceptable liquid carrier, said at least one basic salt being less than 15% by weight, based on the total weight of the second basic liquid component.

2. The composition as claimed in claim 1, wherein said acidic liquid food includes at least one acid selected from the group consisting of citric acid, malic acid, tartaric acid, oxalic acid, succinic acid, fumaric acid, acetic acid, or amyl acid.

3. The composition as claimed in claim 1, wherein content of said acidic liquid food in said first component is 65% to 75% by weight, based on total weight of the first component, and is preferably about 70%.

4. The composition as claimed in claim 1, wherein said first essential oil is selected from the group composed consisting of essential oils extracted from: anise, orange, basil, bergamot, birch, cajeput, chamomile, camphor, cinnamon, cedarwood, cypress, citronella, eucalyptus, fennel seeds, clove leaves, geranium, juniper berry, incense, hyssop, lavender, hybrid lavender, lemongrass, lemon, marjoram, malaleuca alternifolia, tangerine, melissa, mint, myrrh, oregano, patchouli, scotch pine, mugo pine, grapefruit, rose, rosemary, sage, sandalwood, thyme, ylang-ylang, ginger, or a mixture thereof.

5. The composition as claimed in claim 1, wherein content of said at least one first essential oil is 0.01% to 1% by weight, based on the total weight of the first component.

6. The composition as claimed in claim 1, wherein said first component has the following composition:
69.9% lemon juice;
0.1% orange essential oil; and
30% distilled water.

7. The composition as claimed in claim 1, wherein content of said basic salt in said second component is 5% to 10% by weight, based on the total weight of the second component.

8. The composition as claimed in claim 1, wherein said at least one second essential oil is selected from the group consisting of anise, sweet orange, bitter orange, basil, bergamot, birch, cajeput, chamomile, camphor, cinnamon, cedarwood, cypress, citronella, eucalyptus, fennel seeds, clove leaves, geranium, juniper berry, incense, hyssop, lavender, hybrid lavender, lemongrass, lemon, marjoram, malaleuca, tangerine, melissa, mint, myrrh, oregano, patchouli, scotch pine, mugo pine, grapefruit, rose, rosemary, sage, sandalwood, thyme, ylang-ylang, ginger, or a mixture thereof.

9. The composition as claimed in claim 1, wherein content of said second essential oil is 0.005% to 1% by weight, based on the total weight of the second component.

10. The composition as claimed in claim 1, wherein said second component has the following composition:
7.7% sodium bicarbonate;
0.019% thyme essential oil;
0.055% eucalyptus essential oil;
0.055% mint essential oil; and
92.171% distilled water.

11. A method of cosmetic treatment of an oral cavity, comprising:
providing a composition comprising,
a first acidic liquid component, and
a second basic liquid component,
wherein said first acidic liquid component and said second basic liquid component are designed for separate use,
wherein said first acidic liquid component consists of a solution including an acidic liquid food, at least one first essential oil and an orally acceptable liquid carrier said acidic liquid food being more than 60% by weight, based on the total weight of said first acidic liquid component, and
wherein said second basic liquid component consists of a solution including at least one basic salt at least one second essential oil and an orally acceptable liquid carrier, said at least one basic salt being less than 15% by weight, based on the total weight of the second basic liquid component; and
applying the composition to the oral cavity.

12. The method as claimed in claim 11, wherein the step of applying the composition comprises applying a predetermined amount of said first component during a first oral cavity rinsing step of a predetermined duration and then ejecting said first component, and further comprises applying a predetermined amount of said second component during a second rinsing step of a predetermined duration and then ejecting said second component, said second rinsing step being subsequent to the first rinsing step.

13. The method as claimed in claim 12, wherein said predetermined amounts of the first component and second component as used for the first rinsing step is of 5 ml to 20 ml, said predetermined durations of said first rinsing step and second rinsing step being of 20 seconds to 40 seconds.

14. A kit for oral hygiene comprising:
a first container containing a first acidic liquid component consisting of a solution including an acidic liquid food, at least one first essential oil and an orally acceptable liquid carrier, said acidic liquid food being more than 60% by weight, based on the total weight of said first acidic liquid component,
a second container containing a second basic liquid component consisting of a solution including at least one basic salt, at least one second essential oil and an orally acceptable liquid carrier, said at least one basic salt being less than 15% by weight, based on the total weight of the second basic liquid component.

15. The kit as claimed in claim 14, wherein content of said acidic liquid food in said first component is 65% to 75% by weight, based on total weight of the first component.

16. The kit as claimed in claim 14, wherein content of said at least one first essential oil is 0.01% to 1% by weight, based on the total weight of the first component.

17. The kit as claimed in claim 14, wherein said first component consists of 69.9% lemon juice, 0.1% orange essential oil, and 30% distilled water.

18. The kit as claimed in claim 14, wherein content of said basic salt in said second component is 5% to 10% by weight, based on the total weight of the second component.

19. The kit as claimed in claim 14, wherein content of said second essential oil is 0.005% to 1% by weight, based on the total weight of the second component.

20. The kit as claimed in claim 14, wherein said second component consists of 7.7% sodium bicarbonate, 0.019% thyme essential oil, 0.055% eucalyptus essential oil, 0.055% mint essential oil, and 92.171% distilled water.
